(19) United States
(12) Patent Application Publication
(10) Pub. No.: US 2025/0127442 A1
(43) Pub. Date: Apr. 24, 2025

(54) BLOOD COLLECTION SYSTEM

(71) Applicant: BD KIESTRA B.V., Drachten (NL)

(72) Inventors: Daniel James Robertson, Leeuwarden (NL); Samer Ahmed, Assen (NL); John Scott Wheeler, Cockeysville, MD (US)

(73) Assignee: BD KIESTRA B.V., Drachten (NL)

(21) Appl. No.: 18/685,588

(22) PCT Filed: Sep. 6, 2022
(86) PCT No.: PCT/US2022/042637

§ 371 Date: Feb. 22, 2024

Related U.S. Application Data
(60) Provisional application No. 63/241,310, filed on Sep. 7, 2021.

(51) Int. Cl. A61B 5/15

(52) U.S. Cl. CPC A61B 5/150946; A61B 5/15003; A61B 5/150221

(57) ABSTRACT

A blood collection system for determining an accurate blood fill volume in a collection vessel is disclosed herein. The blood collection system includes a flow component and a blood metering device connected to the flow component. The flow component comprises a flow channel having a T-junction. The blood metering device includes a control unit for operating the blood metering device, a barrel configured to align with a neck of a. collection vessel for receiving the neck therewithin. and an adapter including a luer connector at a first end portion thereof for coupling with the control unit and a second end portion thereof for coupling with the barrel. A pressure sensor is disposed in a channel adjacent to and in fluid communication with the T-junction. Methods of determining an accurate blood fill volume in a collection vessel are also disclosed herein.

### CROSS-REFERENCE TO RELATED APPLICATIONS

This application claims priority to and the benefit of U.S. Provisional Application No. 63/241,310 which was filed on Sep. 7, 2021, and is incorporated by reference herein in its entirety.

### TECHNICAL FIELD

The present invention relates to blood collection system, and more particularly, to blood collection systems configured to draw blood from a patient and to fill a culture bottle with an accurate predetermined amount of blood.

### BACKGROUND

During blood collection for blood cultures from patients in hospital or other settings, it is important to provide the blood culture bottles with a targeted amount of blood to ensure that the drawn volume is neither too large nor too small, since inoculating the blood culture with an undersized and oversized sample can adversely affect the accuracy of the results of the blood culture analysis. At this moment, the only feedback to the medical personnel (typically) drawing blood from a patient is visually monitoring the fluid level in the blood culture bottle during blood draw and discontinuing collection when the fill volume is determined to have been reached.

Currently, the medical personnel make this determination visually. The blood culture bottle has a scale of volume measures on the bottle or the bottle label. Often, the medical personnel are required to mark the target filling volume for the blood on the side of the bottle. In practice, this method is susceptible to error. When a medical professional is drawing blood into the blood culture bottle, the medical personnel may not hold the bottle in a precisely vertical orientation, making it difficult or even impossible to determine the actual volume of the blood collected and making it likely that the target volume of the blood is not obtained. Another issue that can affect the accuracy of the volume of blood drawn is the lack of uniform instructions for how to properly inoculate the blood culture bottle with the target amount of blood. Also, the needs of the patient (who may have difficulties during the blood draw that might distract the medical personnel from accurately monitoring the blood draw) might adversely affect the accuracy of the volume of blood drawn by the medical personnel.

Successfully culturing and detecting a bacteria that has infected a patient is highly dependent on collecting the bacteria in the blood sample taken from the patient. The probability of having bacteria in the blood sample increases with an increase in the volume of blood collected. Therefore, collecting the target volume called for in a blood culture bottle, one example of which is a BACTEC™ culture bottle, with precision, is very important.

As noted above, currently, the medical personnel collecting the blood sample must visually determine when the correct volume of blood has been drawn and collected in the culture bottle, and stop the collection precisely at that point to avoid over-filling the blood culture bottle. Therefore, methods and apparatus for collecting blood that can ensure a target volume of blood is accurately collected continue to be sought.

### BRIEF SUMMARY

Described herein is a blood collection system for determining an accurate blood fill volume in a collection vessel. The blood collection system includes a flow component and a blood metering device connected to the flow component. The flow component comprises a flow channel having a T-junction. The blood metering device includes a control unit for operating the blood metering device, a barrel configured to align with an opening, such as a neck, of a collection vessel for receiving the neck therewithin, and an adapter including a luer connector at a first end portion thereof for coupling with the control unit and a second end portion thereof for coupling with the barrel. The control unit includes a housing defining a blood flow conduit for blood from a patient to flow therethrough, a valve disposed within the blood flow conduit, and an electronic module disposed within an electronic compartment of the housing. The valve is adapted to allow the blood from the patient to flow through the blood flow conduit when the valve is in an open position and to stop the blood from the patient from flowing through the blood flow conduit when the valve is in a closed position. A pressure sensor is disposed in a channel adjacent to and in fluid communication with the T-junction.

Also described herein is a method for determining an accurate blood fill volume in a collection vessel with the blood collection system described above. The method includes connecting the blood metering device to the blood collection vessel such that the blood metering device is in fluid communication with the blood collection vessel, inputting a predetermined fill volume to the blood metering device, determining a gas pressure in the collection vessel with the valve closed, determining a target gas pressure in the collection vessel at the predetermined fill volume based on the inputted predetermined fill volume and determined gas pressure, determining an expected amount of time to reach the predetermined fill volume based on the determined target gas pressure, closing the valve when the expected amount of time to reach the predetermined fill volume is reached, continuously determining a gas pressure in the collection vessel, continuously comparing the determined gas pressure in the collection vessel with the determined target gas pressure, and closing the valve when the determined gas pressure is equal to the determined target gas pressure. The determined target gas pressure in the collection vessel indicates that a target volume of blood has entered the collection vessel.

Also described herein is an alternate method for determining an accurate blood fill volume in a collection vessel with the blood collection system described above. The method includes connecting the blood metering device to the blood collection vessel such that the blood metering device is in fluid communication with the blood collection vessel, inputting a predetermined fill volume to the blood metering device, determining a target gas pressure in the collection vessel at the predetermined fill volume, opening the valve to allow blood drawn from a patient to flow, measuring pressure at a T-junction of the flow component as the blood flows therethrough, determining gas pressure in the collection vessel based on the measured pressure at the T-junction of the flow component, comparing the determined gas pressure in the collection vessel with the determined target gas pressure, closing the valve when the determined gas pressure is equal to the determined target gas pressure. The determined target gas pressure in the collection vessel indicates that a target volume of blood has entered the collection vessel.

These and other aspects of the present invention will be better understood in view of the drawings and following detailed description.

### BRIEF DESCRIPTION OF DRAWINGS

FIG. 1 is a blood collection system according to an embodiment of the present invention.

FIG. 2 is a cross-sectional view of a blood metering device of the blood collection system of FIG. 1.

FIG. 3 is a cross-sectional view of a control unit of the blood metering device of FIG. 2.

FIG. 4 is a flow chart of a method for determining an accurate blood fill volume in a collection vessel, according to an embodiment of FIG. 1.

FIG. 5 is a flow chart of a method for determining an accurate blood fill volume in a collection vessel, according to an alternate embodiment.

FIG. 6 is a graph of blood dynamic viscosity (shear rate vs. blood viscosity).

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The blood metering device described herein collects blood from a patient and fills a blood collection vessel or collection bottle, which the device is attached, with an accurate volume of blood. The collection bottle is any suitable container for receiving a blood sample. One example is a blood collection tube such a BD Vacutainer® tube. BD Vacutainer is a registered trademark of Becton, Dickinson and Company. Another example is a blood culture bottle such as the BACTEC bottle described above. The blood metering device provides at least one of: 1) an indication when a target volume of blood has passed through the device and into the collection bottle: or 2) an automatic shut off when a target volume of blood has passed through the device and into the collection bottle.

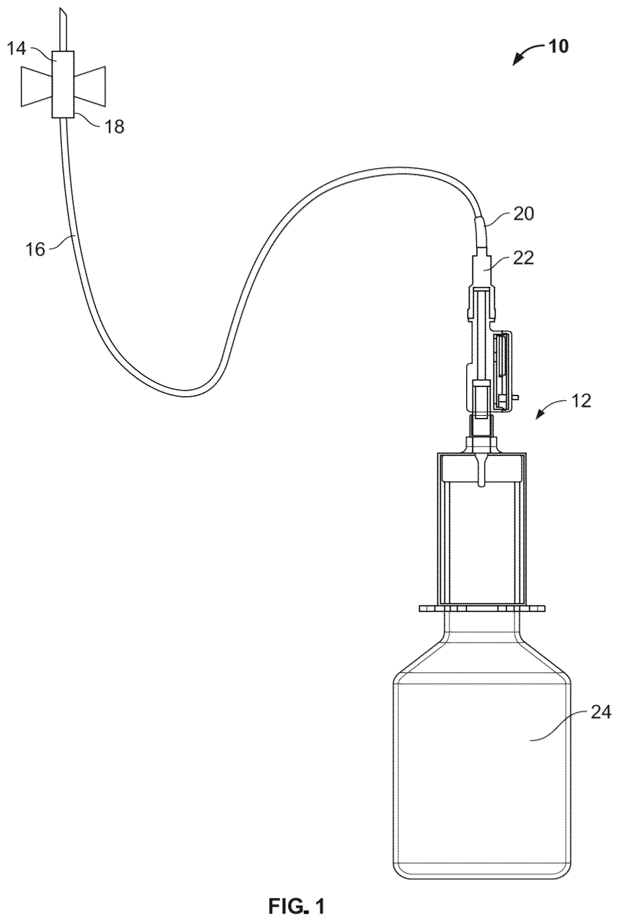

FIG. 1 illustrates a blood collection system 10 comprising a blood metering device 12 according to an embodiment of the present technology. The blood collection system 10 includes a butterfly needle 14, tubing 16, and a blood metering device 12 connected to the butterfly needle 14 via the tubing 16. Specifically, the butterfly needle 14 is connected to a first end 18 of the tubing 16 and the blood metering device 12 is connected to a second end 20 of the tubing 16 via a tubing connector 22.

Although in the illustrations herein, the blood collection system 10 is illustrated with a collection vessel 24 resting on its bottom surface, the blood collection system is not required to be in this precise orientation. If the collection vessel 24 is held in an upright but tilted position, the blood collection will also be tilted from vertical. Sine the pressure in the headspace of the culture bottle is monitored by the blood collection system 10, the blood collection system 10 is held in a position that permits communication of the blood collection system 10 with the headspace of the culture bottle to make the necessary measurements.

Therefore, directional terms, such as top and bottom, are referenced to an orientation in which the blood metering device 12 is connected to a collection vessel 24 placed on a flat surface. However, the present invention is not thereby limited to use in any particular orientation.

During the process of collecting a blood sample from a patient, the butterfly needle 14 is used to pierce a vein or an artery of the patient. Driven by the vacuum pressure created by the collection vessel 24, blood from the patient is directed toward the collection vessel 24 through the tubing 16. A flow of blood is collected in the collection vessel 24. Along the way, the blood passes through the blood metering device 12.

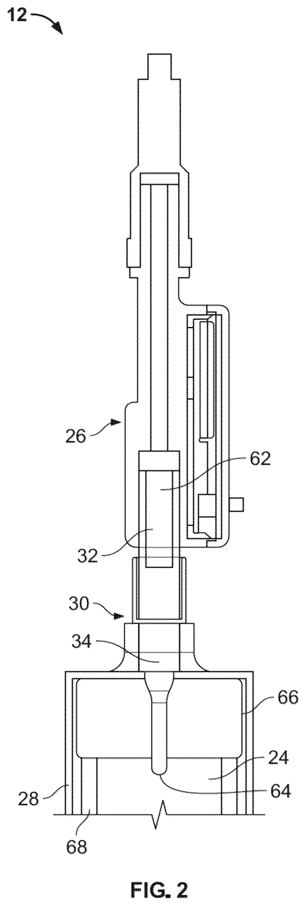

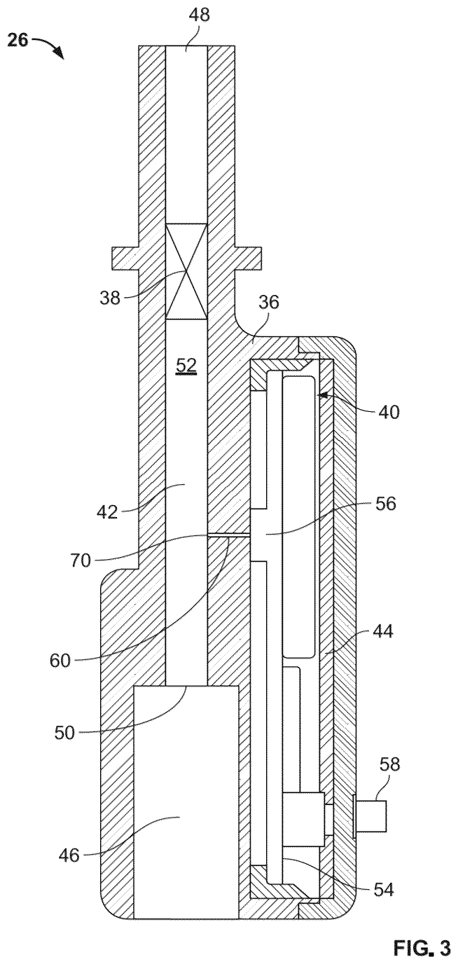

Referring to FIGS. 2 and 3, the blood metering device 12 includes a control unit 26, a barrel 28, and an adapter 30 configured to connect the control unit 26 and the barrel 28. Specifically, the control unit 26 is connected to a first end portion 32 of the adapter 30, and the barrel 28 is connected to a second end portion 34 of the adapter 30. Thus, the adapter 30 is connected to the control unit 26 and barrel 28 such that the control unit 26 and barrel 28 are located on top and bottom of the adapter 30, respectively. The control unit 26, a barrel 28, and an adapter 30 are all in fluid communication with each other such that blood drawn from a patient flows to the collection vessel 24.

Referring particularly to FIG. 3, the control unit 26 includes a housing 36, a valve 38, and an electronic module 40 disposed within the housing 36. The housing 36 defines a blood flow conduit 42 for blood from a patient to flow therethrough to the collection vessel 24, an electronic compartment 44 for accommodating the electronic module 40 therewithin, and a connection cavity 46 for engaging and coupling with the adapter 30.

The blood flow conduit 42 extends between a first open end 48 and a second open end 50 thereof. If the collection vessel 24 is supported by a flat, horizontal surface, the orientation of the blood flow conduit 42 is approximately vertical. As can be understood with reference to FIGS. 1-3, the blood flow conduit 42 is connected to and is in fluid communication with the tubing 16, via the tubing connector 22, at the first open end 48 of the blood flow conduit 42. The blood flow conduit 42 defines a passageway 52 therewithin for the drawn blood to travel therethrough and to the collection vessel 24. The passageway 52 of the blood flow conduit 42 extends between the first open end 48 of the blood flow conduit 42 and the second open end 50 of the blood flow conduit 42. The connection cavity 46 is connected to the blood flow conduit 42 at the second open end 50 thereof. As stated above, the connection cavity 46 is dimensioned and configured to tightly engage and couple with the adapter 30.

The valve 38 is positioned within the blood flow conduit 42. The valve 38 is integrated with a valve actuator for controlling blood flow from a patient. Valves 38 suitable for this purpose are well known to one skilled in the art and are not described in detail herein. Suitable valves for the blood metering device 12 include a pinch valve, diaphragm valves, ball valves, slide valves, check valves, release valves, etc.

The electronic module 40 is disposed within the electronic compartment 44 and includes a printed circuit board (PCB) 54 for controlling various components in the blood metering device 12, a pressure sensor 56 connected to the PCB 54, a user input control 58, a valve actuator for controlling the valve 38, and a battery.

The PCB 54 includes a microcontroller having a processor and a memory therewithin, and other electronics necessary to facilitate the operation of the various components of the blood metering device 12. For example, the processor can actuate the valve actuator to open the valve 38 to commence the blood collection process and close the valve 38 once the predetermined fill volume has been filled in the collection vessel 24. The memory stores information therein that controls the operation of the blood metering device 12. Non-limiting examples of such information includes total blood volume that passes through the blood metering device 12 (i.e., the predetermined fill volume), the maximum duration of the blood draw (after which time the blood metering device 12 terminates further collection of the blood from the patient), and changes in blood flow rate from the patient indicative of vein collapse. In addition, the microcontroller provides blood collection process information to the user via an LED (not shown) or other suitable indicator/display installed on the blood metering device 12. For example, the LED provides an indication of blood volume that has passed through the blood metering device 12 (illustrated as colored light) and has reached the predetermined fill volume. Other indicators that the predetermined fill volume has been received by the collection vessel 24 include sensory alerts such as vibration alert or audible signal.

The user input control 58 allows a user to input information (e.g., predetermined fill volume) for the microcontroller to compute and determine necessary information for operating the blood collection system 10. In addition, the audio volume of the blood metering device 12 may be adjusted via the user input control 58.

The valve actuator controls the flow of blood collected from the patient by keeping the valve 38 closed when blood draw from the patient commences. After blood draw is commenced, the valve actuator receives a signal from the microcontroller indicating that blood flow has started. In response to such signal, the valve actuator gradually causes the valve 38 to open. In addition, the valve actuator is programmed, via the microcontroller, to open the valve 38 in a manner that mitigates hemolysis of the blood flowing through the blood flow conduit 42.

Once the predetermined fill volume of blood has been filled in the collection vessel 24, the microcontroller again sends a signal to the valve actuator indicating that the predetermined fill volume has been reached. In response to such signal, the valve actuator causes the valve 38 to close and automatically shut the blood metering device 12 off.

Suitable valve actuators are well known to one skilled in the art and are not described in detail herein. Such actuators include moving magnet actuators, micro actuators, solenoids, paired magnets, DC motors, etc. that, in response to a signal, cause the valve 38 to open or close.

Referring again to FIG. 3, a narrow channel 60 is defined in the housing 36 of the control unit 26 and connects the blood flow conduit 42 and the pressure sensor 56 disposed within the electronic compartment 44 of the housing 36. The channel 60 allows the pressure sensor 56 to measure the gas pressure in the collection vessel 24 when the valve 38 is closed, which stops the blood from flowing through the blood metering device 12 and to the collection vessel 24.

Referring again to FIGS. 2 and 3, as stated above, the adapter 30 is configured to connect to both the control unit 26 and the collection vessel 24. Specifically, the adapter 30 includes a luer connector 62 at the first end portion 32 for coupling with the control unit 26 and a needle (not shown) contained in a rubber sheath 64 at the second end portion 34 for coupling with the collection vessel 24. The luer connector 62 is inserted into the connection cavity 46 of the control unit 26 such that the luer connector 62 is tightly engaged with the connection cavity 46 and connected to the control unit 26. The needle in the adapter 30 is used to pierce a cap 66 of the collection vessel 24 and to fill blood drawn from the patient in the collection vessel 24. Thus, the blood metering device 12 is adapted to be fluidically coupled to the collection vessel 24. While, in the depicted embodiment, the adapter 30 is connected to the control unit 26 via the luer connector 62, alternatively, the adapter 30 may be coupled to the control unit 26 by other conventional coupling means (e.g. threaded connection, snap connection, etc.).

The barrel 28 is configured to be connected to the adapter 30 at the second end portion 34 thereof. The barrel 28 is designed and dimensioned such that once the needle pierces the cap 66 of the collection vessel 24, the barrel 28 aligns with a neck 68 of the collection vessel 24 such that the neck 68 is received within the barrel 28.

The blood metering device 12 is made of one or more materials having suitable properties for a desired application, including strength, weight, rigidity, etc. Plastic (e.g., polypropylene, polyethylene, etc.) is preferred for the housing 36 and barrel 28 of the blood metering device 12.

The blood metering device 12 according to the present technology is configured to monitor, measure, and control the fill level of the collection vessel 24 (e.g., the volume of the blood introduced into the collection vessel) by measuring the gas pressure in the collection vessel 24 using the pressure sensor 56. Specifically, as the blood enters the collection vessel 24, the gas pressure in the collection vessel 24 increases, which is caused by addition of the blood to the collection vessel 24, which reduces the gas volume in the container. Thus, the blood collection system 10 measures the vacuum pressure in the collection vessel before and, during the blood collection process, estimates the amount of blood added to the collection vessel 24 at a specific time. This estimation of the amount of blood added to the collection vessel 24 may be calculated using Boyle's law, which states that the pressure of a given quantity of gas varies inversely with its volume. This relationship is linear, if pressure on a gas doubles, its volume decreases by one-half. The estimation may be determined using Boyle's law since the total volume of the collection vessel 24 and the total amount of gas in the collection vessel 24 do not change.

In the device described herein, a valve 38 is provided in order to stop the flow of sample into the collection vessel 24 in order to measure the internal pressure of the collection vessel from which the fill volume can be determined.

The valve 38 is located between a patient (not shown) and the narrow channel 60 for that is in communication with pressure sensor 56 in the control unit 26. The location of the valve 38 allows the pressure sensor 56 to measure an initial pressure at the narrow channel 60 of the control unit 26. The pressure sensor 56 is disposed in an airtight chamber, so it is placed in a closed system, and the only influence on the pressure sensor measurement is the fluid pressure of the blood in the passageway, which increases as the blood enters the collection vessel 24 and increases the pressure in the headspace of the collection vessel 24. When the blood metering device 12 is initially connected to the collection vessel (24 in FIG. 1) to commence the blood collection process, the valve 38 is initially closed.

A membrane (not shown) may be disposed between the pressure sensor 56 and the blood flowing in the narrow channel 60 to ensure the sterility of blood sample from the patient and that the sample is not contaminated by impurities such as bacteria that might be resident in non-sterile areas of the pressure sensor 56. The membrane allows pressure to be transmitted from the sterile area to the pressure sensor 56 but to block living organisms (e.g., bacteria, fungi, etc.) from travelling from the non-sterile pressure sensor area to the sterile area. The membrane can be air-permeable, or alternately flexible and non-permeable.

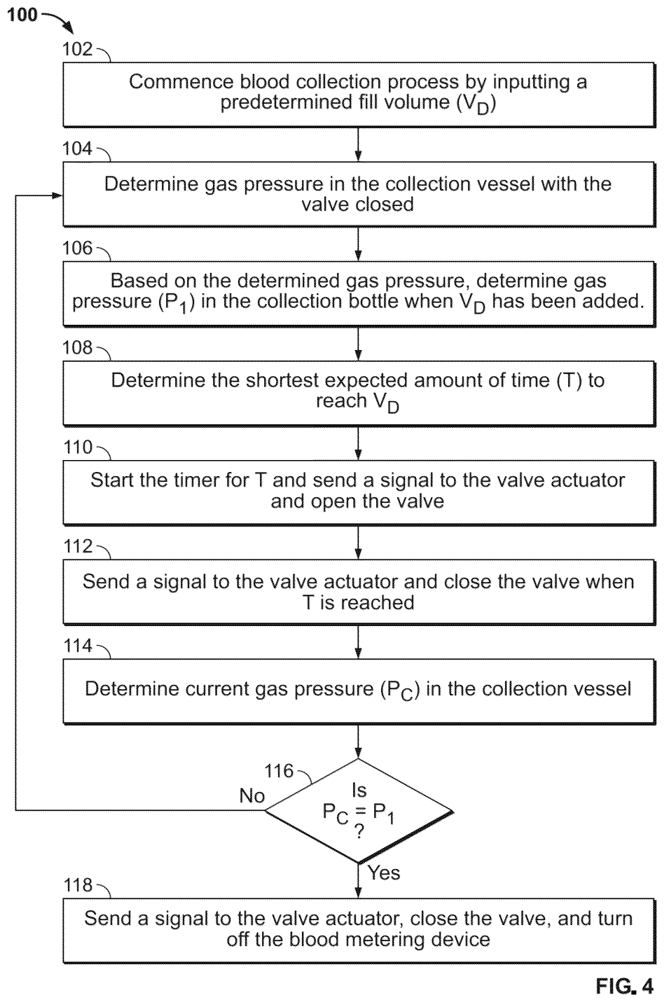

FIG. 4 illustrates a method 100 of determining an accurate blood fill volume in a blood collection vessel 24 using the blood collection system 10 in accordance with the first embodiment of the present technology. At step 102, a blood collection process is commenced by inputting (or selecting) a predetermined desired fill volume (V_{D}) to the blood metering device 12 by a user. Then, at step 104, with the valve 38 closed, the gas pressure in the collection vessel 24 is measured using the pressure sensor 56 disposed in the control unit 26. By using the pressure sensor fill algorithm based on Boyle's law, the user input and the measured gas pressure, the microcontroller of the blood metering device 12 determines the target pressure (P_{I}) of the gas in the collection vessel 24 that will indicate that the desired fill volume (V_{D}) has been added into the collection vessel at step 106. At step 108, using the pressure sensor fill algorithm and based on the target pressure, the shortest expected amount of time (T) to reach V_{D} is calculated. At step 110, a timer for T is set, and a signal is sent to the valve actuator to open the valve 38 to permit blood drawn from a patient to flow to the collection vessel 24. When T has reached, at step 112, a signal is sent to the valve actuator to close the valve 38 to stop blood drawn from a patient to flow to the collection vessel 24. At step 114, the pressure sensor 56 measures the gas pressure in the collection vessel 24 and transmits current pressure data to the microcontroller of the blood metering device 12. At step 116, the blood metering device 12 compares the received gas pressure data (e.g., current gas pressure in the collection vessel) with target pressure P_{I}. If the blood metering device 12 determines that the measured gas pressure (P_{C}) in the collection vessel 24 at the current fill volume is equal to the target pressure P_{I}, a signal is sent to the valve actuator to keep the valve 38 closed and the blood metering device 12 turns of automatically at step 118. If, however, the blood metering device 12 determines P_{C} is less than P_{I} at step 116, the steps 104-116 are repeated until P_{C} is equal to P_{I}.

In an alternate embodiment, the fill level of the collection vessel 24 (e.g., volume of the blood in the collection vessel) is determined by measuring the gas pressure at the T-junction point (P_{T}) 70 (shown in FIG. 3) located in the blood flow conduit 42 of the control unit 26. Thus, rather than closing the valve 38 repeatedly and stopping blood flow to measure the gas pressure in the collection vessel 24 during the blood collection process as described in the first embodiment, an algorithm based on physical modelling of the fluid flow system is used to infer the gas pressure in the collection vessel 24 based on the gas pressure at the T-junction point 70. The fluid pressure at the T-junction point 70 is continuously monitored and measured as the blood sample flows through the passageway 52 and into the collection vessel 24. In one optional embodiment, the correlation between the measured gas pressure at the T-junction point 70 and the gas pressure in the collection vessel 24 (from which the volume of the blood sample that has entered the collection vessel 24 is determined) will be described in greater detail below.

The blood flow path comprises various components that channel the blood from collection and into the collection vessel. For example, and as illustrated, blood flows from a patient and travels through the butterfly needle 14, tubing 16, pressure sensor 56, blood flow conduit 42, needle (not shown) contained in the rubber sheath 64 and into the collection vessel 24. Although a particular selection and arrangement of components is described, contemplated herein are other components that can be arranged to convey blood collected from a patient to a blood collection vessel, wherein the fluid pressure in the passageway is measured by a sensor to determine the amount of blood that has entered the collection vessel.

Because of the effect of the various components in the blood fluid pathway, the measured pressure of the fluid by the pressure sensor 56 may be substantially different from the pressure in the collection vessel 24. For example, the pressure of the blood drawn from the patient drops from atmospheric pressure at the patient to 0.5 bar (50 kilopascals) to 0.75 bar (75 kilopascals) absolute pressure of the collection vessel 24. Due to this pressure drop, the gas pressure in the collection vessel (P_{C}) can differ substantially from what the pressure sensor 56 measures as the blood travels through the passageway 52 of the blood flow conduit 42.

The variables for determining the pressure at any given point in the blood flow pathway include the blood viscosity, the diameter of each of the various component of the blood flow pathway (e.g., blood flow conduit 42, the passageway 52), the flow rate, and the absolute pressure in the collection vessel 24. None of these variables are known by the control unit 26. Optionally, these variables can be related to the pressure drop and may be determined, for example, by using the Hagen-Poiseuille equation.

To accurately set the fill volume (V_{D}) of the collection vessel 24, the control unit 26 is programmed to determine an accurate estimate of the value P_{C} at both the start and at the end of the blood fill process. However, because the pressure sensor 56 is configured to measure the gas pressure at the passageway 52 of the blood flow conduit 42, described herein is a novel and inventive way for the control unit 26 to determine an accurate estimate of P_{C} at both the start and end of the blood fill process.

In one embodiment, the estimate of P_{C} is based on the relationship between P_{C} and P_{T}. Such a relationship is optionally derived by the application of the Hagen-Poiseuille equation while making assumptions about the sheer rate of blood in the device, the linear flow behavior of the fluid-carrying components during the blood draw, and the blood pressure in the patient.

In one example, resistances to flow can be modelled using the Hagen-Poiseuille equation (Δp=8μ LQ/(πr⁴)). In this equation the variables are:
Δp is the pressure difference between the two ends;
L is the length of fluid flow component;
μ is the dynamic viscosity of the fluid (e.g., the blood);
Q is the volumetric flow rate of the fluid in the fluid flow component;
r is the pipe radius; and
A is the cross-sectional area of the fluid flow channel in the fluid flow component.

For a draw with a given length of a fluid flow component (e.g., tubing for purposes of this example) L, r, and π remain constant provided that the fluid flow channel of the relevant component is not being pinched during the draw. Given these assumptions, the equation can be simplified to:

$$Δ p = c * μ Q ; \qquad ( 1 )$$

$$where c = 8 L / ( π {r}^{4} ) . \qquad ( 2 )$$

The flow resistance R of a given segment of tubing (i.e., a flow component) can then be defined as:

$$R = Δ p / Q . \qquad ( 3 )$$

Combining these:

$$R = Δ p / Q = c * μ Q / Q = c μ \qquad ( 4 )$$

For the first section of blood collection components, between the patient and the valve:

$$R 1 = c 1 * μ1 \qquad ( 5 )$$

$$R 2 = c 2 * μ 1 \qquad ( 6 )$$

During the blood fill process, the collection vessel pressure (P_{c}) may be estimated using the following equation:

$$Pc = Pr + ( R 2 / R 1 ) * ( {P}_{T} - {P}_{ambient} ) \qquad ( 7 )$$

where R1 is the flow resistance between the patient and the valve 38, R2 is the flow resistance between the valve 38 and the inside of the collection vessel 24, and P_{ambient} is the ambient air pressure. (R2/R1) is assumed to be a constant during the duration of the blood draw.

Although not wishing to be held to a particular theory, this assumption is based on the fact that there is the 4^{th} power dependence on r. Therefore, resistance of a section of tubing will be dominated by the components with the narrowest flow channels (i.e., smallest r) in that section. In the context of the blood collection system described herein, the narrowest flow channel is the needle into the patient's arm in the case of R1, and the needle piercing the septum of the collection vessel in the case of R2.

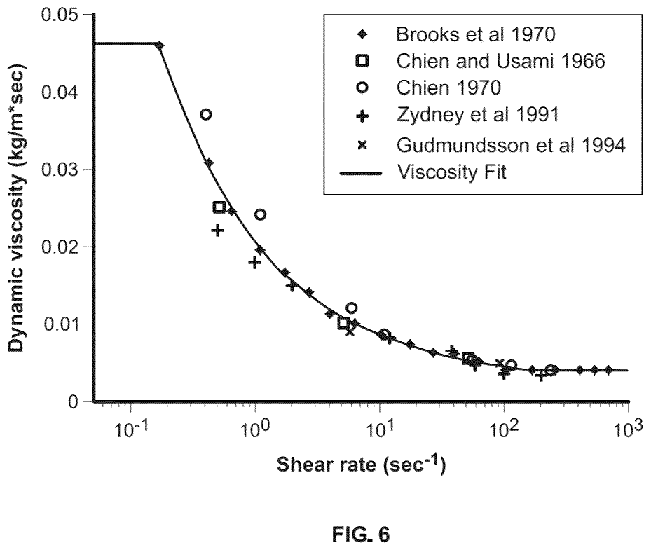

In one embodiment in which the patient needle flow channel radius is approximately equal to the radius of the flow channel for the collection vessel septum needle, the sheer rate 7′ will be the same for both R1 and R2. Also, for a 1 ml/s fill rate (of the collection vessel) with a 0.5 mm inner diameter needle, the sheer rate is calculated to be 5093 per second. For tubing with a 1 mm inner diameter, this value falls to a sheer rate of 2546 per second. The dynamic viscosity in this region is substantially constant as illustrated in FIG. 6.

This means the equation can be simplified to the following, which reveals that the value of R2/R1 is a constant.

$$( R 2 / R 1 ) = ( c 2 * μ ) / ( c 1 * μ ) = c 2 / c 1 = constant \qquad ( 8 )$$

By using the equation:

$$( R 2 / R 1 ) = ( Pc - PT ) / ( PT - {P}_{ambient} ) \qquad ( 9 )$$

(R2/R1) may be estimated immediately after (e.g., one second) opening the valve 38 for the first time after connecting the blood collection system 10 to the patient. Since the pressure sensor 56 is close to the collection vessel 24, it is also possible to use a pressure spike at the pressure sensor 56 to infer (R2/R1) when blood first starts entering the collection vessel 24. P_{ambient} is equal to the patient blood pressure (e.g., 1 bar or 100 kilopascals) or may be measured prior to the blood draw.

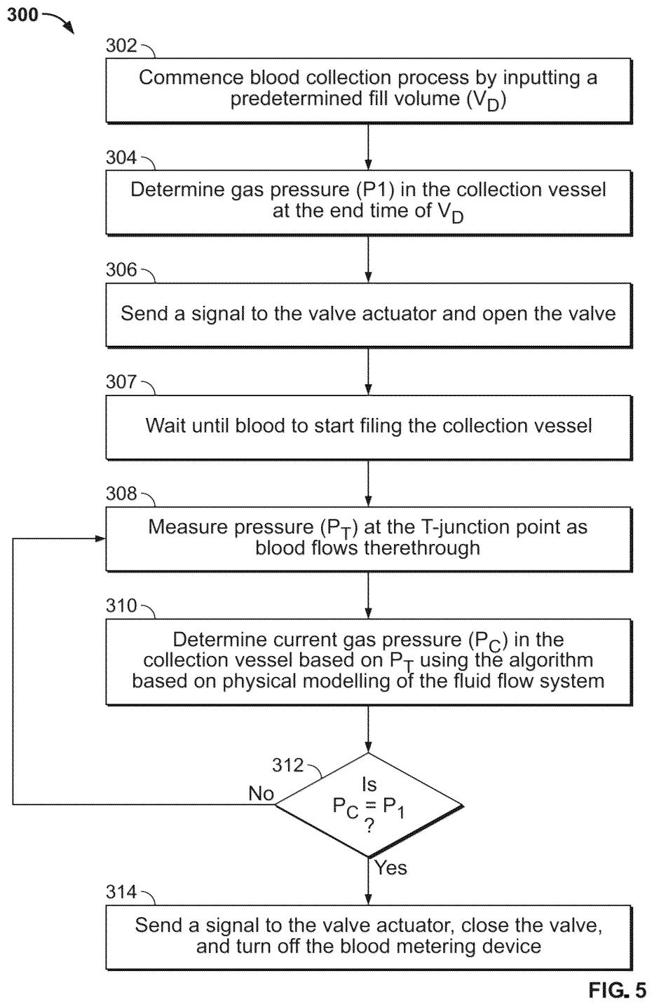

FIG. 5 illustrates a method 300 of determining an accurate blood fill volume in a blood collection vessel 24 using the blood collection system 10 in accordance with the alternate embodiment of the present technology. At step 302, a blood collection process is commenced by inputting (or selecting) a predetermined fill volume (V_{D}) to the blood metering device 12 by a user. By using the pressure sensor fill algorithm based on the Boyle's law and the user input, the microcontroller of the blood metering device 12 determines the target pressure (P_{I}) of the gas in the collection vessel 24 that will indicate that the desired fill volume at (V_{D}) has been added into the collection vessel at step 304. At step 306, a signal is sent to the valve actuator to open the valve 38 to permit blood drawn from a patient to flow through the blood metering device 12. Thereafter, at step 307, the blood collection system 10 waits until the blood drawn from the patient starts filling the collection vessel 24. At step 308, pressure (P_{T}) at the T-junction point 70 is measured as blood flows therethrough. Then, at step 310, the microcontroller of the blood metering device 12 determines the gas pressure (P_{C}) in the collection vessel 24 based on P_{T} and using the algorithm based on physical modelling of the fluid flow system. At step 312, the blood metering device 12 compares P_{C} with P_{I}. If the blood metering device 12 determines that P_{C} is equal to P_{I}, a signal is sent to the valve actuator to close the valve 38 to stop the blood flow, and the blood metering device 12 turns off automatically at step 314. If, however, the blood metering device 12 determines that P_{C} is less than P_{I} at step 312, the valve 38 is kept open to permit the blood to travel to the collection vessel 24, and the steps (308-312) are performed again. The steps (308-312) are repeated until P_{C} is equal to P_{I}.

Described herein is a blood collection system having a flow component comprising a flow channel extending between a first end and a second end of the flow component; a butterfly needle connected to the first end of the flow component; and a blood metering device connected to the second end of the flow component, the blood metering device having a control unit, the control unit including a housing defining a blood flow conduit for blood from a patient to flow therethrough, a valve disposed within the blood flow conduit, the valve adapted to allow the blood from the patient to flow through the blood flow conduit when the valve is in an open position and to stop the blood from the patient from flowing through the blood flow conduit when the valve is in a closed position, and an electronic module disposed within an electronic compartment of the housing. The system also has a barrel configured to align with a neck of a collection vessel for receiving the neck therewithin and an adapter including a luer connector at a first end portion thereof for coupling with the control unit and a second end portion thereof for coupling with the barrel.

In one aspect, the system has a connection cavity is defined within the housing of the control unit for engaging and coupling with the adapter. In a further aspect, the blood flow conduit defines a passageway therewithin for the blood from the patient to travel therethrough and to the collection vessel. In any of the above aspects, the electronic module includes a printed circuit board for controlling components in the blood metering device, and a pressure sensor connected to the printed circuit board, and a user input control. In any of the above aspects, a channel is defined in the housing of the control unit and connects the blood flow conduit and a pressure sensor disposed within the electronic compartment of the housing. In any of the above aspects, the channel allows the pressure sensor to measure a gas pressure in the collection vessel when the valve is closed, which stops the blood from flowing through the blood metering device and to the collection vessel.

In any of the above aspects, the control unit, barrel, and adapter are all connected and in fluid communication with each other such that blood from the patient flows to the collection vessel.

In a further aspect, described herein is a method for determining an accurate blood fill volume in a collection vessel, the method comprising: providing a blood collection system having a flow component with a flow channel extending therethrough between a first end and a second end; a butterfly needle connected to the first end of the flow component; and a blood metering device connected to the second end of the flow component, the blood metering device having a control unit, the control unit including a housing defining a blood flow conduit for blood from a patient to flow therethrough, a valve disposed within the blood flow conduit, and an electronic module disposed within an electronic compartment of the housing. In a further aspect the system has a barrel configured to align with a neck of a blood collection vessel to receive the neck therewithin; and an adapter including a luer connector at a first end portion thereof for coupling with the control unit and a second end portion thereof for coupling with the barrel. The method further comprises connecting the blood metering device to the blood collection vessel such that the blood metering device is in fluid communication with the blood collection vessel; inputting a predetermined fill volume to the blood metering device; determining a gas pressure in the collection vessel with the valve closed. According to the method, based on the inputted predetermined fill volume and determined gas pressure, a target gas pressure is determined in the collection vessel at the predetermined fill volume. Based based on the determined target gas pressure, an expected amount of time to reach the predetermined fill volume is determined. According to the method, the valve is closed when the expected amount of time to reach the predetermined fill volume is reached. The gas pressure in the collection vessel is continuously determined and the determined gas pressure in the collection vessel continuously compared with the determined target gas pressure. The method further comprises closing the valve when the determined gas pressure is equal to the determined target gas pressure, wherein the determined target gas pressure in the collection vessel indicates that a target volume of blood has entered the collection vessel.

In one aspect of the method, a channel is defined in the housing of the control unit and connects the blood flow conduit and a pressure sensor disposed within the electronic compartment of the housing. In a further aspect, the channel allows the pressure sensor to measure the gas pressure in the collection vessel when the valve is closed, which stops the blood from flowing through the blood metering device and to the collection vessel.

Also described herein is a blood collection system having: i) a flow component comprising a flow channel extending between a first end and a second end, the flow channel having a T-junction: ii) a butterfly needle connected to the first end of the flow component; iii) a valve disposed between the butterfly needle and the T-junction of the flow channel; iv) a pressure sensor disposed in a channel adjacent to and in fluid communication with the T-junction; and v) a blood metering device connected to the second end of the flow component, the blood metering device having: i) a control unit, the control unit including a housing defining a blood flow conduit for blood from a patient to flow therethrough and an electronic module disposed within an electronic compartment of the housing; ii) a barrel configured to align with a neck of a collection vessel for receiving the neck therewithin; and iii) an adapter including a luer connector at a first end portion thereof for coupling with the control unit and a second end portion thereof for coupling with the barrel.

In a further aspect, a connection cavity is defined within the housing of the control unit for engaging and coupling with the adapter. In yet another aspect, the blood flow conduit defines a passageway therewithin for the blood from the patient to travel therethrough and to the collection vessel. According to any of the above aspects, the electronic module includes a printed circuit board for controlling components in the blood metering device, the pressure sensor connected to the printed circuit board, and a user input control. According to the above aspects, the channel is defined in the housing of the control unit and connects the blood flow conduit and the pressure sensor. According to the above aspects, the control unit, barrel, and adapter are all connected and in fluid communication with each other such that blood from the patient flows to the collection vessel. According to the above aspects, the channel allows the pressure sensor to measure a gas pressure at the T-junction.

Described herein is a further method for determining an accurate blood till volume in a collection vessel, the method comprising: providing a blood collection system having i) a flow component comprising a flow channel extending between a first end and a second end, the flow component comprising a T-junction; ii) a butterfly needle connected to the first end of the flow component; iii) a valve disposed between the butterfly needle and the T-junction of the flow component; iv) a pressure sensor connected to the flow component at the T-junction; and
v) a blood metering device connected to the second end of the flow component, the blood metering device having: i) a control unit, the control unit including a housing defining a blood flow conduit for blood from a patient to flow therethrough and an electronic module disposed within an electronic compartment of the housing; ii) a barrel configured to align with a neck of a blood collection vessel to receive the neck therewithin; and iii) an adapter including a luer connector at a first end portion thereof for coupling with the control unit and a second end portion thereof for coupling with the barrel. The method further comprises: a) connecting the blood metering device to the blood collection vessel such that the blood metering device is in fluid communication with the blood collection vessel; b) inputting a predetermined fill volume to the blood metering device; c) determining a target gas pressure in the collection vessel at the predetermined fill volume; d) opening the valve to allow blood drawn from a patient to flow; e) measuring pressure at the T-junction of the flow component as the blood flows therethrough; f) determining gas pressure in the collection vessel based on the measured pressure at the T-junction of the flow component; g) comparing the determined gas pressure in the collection vessel with the determined target gas pressure; and h) closing the valve when the determined gas pressure is equal to the determined target gas pressure, wherein the determined target gas pressure in the collection vessel indicates that a target volume of blood has entered the collection vessel.

In a further aspect of the method, a channel is defined in the housing of the control unit and connects the blood flow conduit and a pressure sensor disposed within the electronic compartment of the housing. In yet a further aspect, the channel allows the pressure sensor to measure the gas pressure at the T-junction.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A blood collection system comprising:
a flow component comprising a flow channel extending between a first end and a second end of the flow component;
a butterfly needle connected to the first end of the flow component; and
a blood metering device connected to the second end of the flow component, the blood metering device comprising;
a control unit, the control unit including a housing defining a blood flow conduit for blood from a patient to flow therethrough, a valve disposed within the blood flow conduit, the valve adapted to allow the blood from the patient to flow through the blood flow conduit when the valve is in an open position and to stop the blood from the patient from flowing through the blood flow conduit when the valve is in a closed position, and an electronic module disposed within an electronic compartment of the housing;
a barrel configured to align with a neck of a collection vessel for receiving the neck therewithin; and
an adapter including a luer connector at a first end portion thereof for coupling with the control unit and a second end portion thereof for coupling with the barrel.

2. The blood collection system of claim 1, wherein a connection cavity is defined within the housing of the control unit for engaging and coupling with the adapter.

3. The blood collection system of claim 1, wherein the blood flow conduit defines a passageway therewithin for the blood from the patient to travel therethrough and to the collection vessel.

4. The blood collection system of claim 1, wherein the electronic module includes a printed circuit board for controlling components in the blood metering device, a pressure sensor connected to the printed circuit board, and a user input control.

5. The blood collection system of claim 1, wherein a channel is defined in the housing of the control unit and connects the blood flow conduit and a pressure sensor disposed within the electronic compartment of the housing.

6. The blood collection system of claim 5, wherein the channel allows the pressure sensor to measure a gas pressure in the collection vessel when the valve is closed, which stops the blood from flowing through the blood metering device and to the collection vessel.

7. The blood collection system of claim 1, wherein the control unit, barrel, and adapter are all connected and in fluid communication with each other such that blood from the patient flows to the collection vessel.

8. A method for determining an accurate blood fill volume in a collection vessel, the method comprising:
providing a blood collection system comprising:
a flow component with a flow channel extending therethrough between a first end and a second end;
a butterfly needle connected to the first end of the flow component; and
a blood metering device connected to the second end of the flow component, the blood metering device comprising;
a control unit, the control unit including a housing defining a blood flow conduit for blood from a patient to flow therethrough, a valve disposed within the blood flow conduit, and an electronic module disposed within an electronic compartment of the housing;
a barrel configured to align with a neck of a blood collection vessel to receive the neck therewithin; and
an adapter including a luer connector at a first end portion thereof for coupling with the control unit and a second end portion thereof for coupling with the barrel;
connecting the blood metering device to the blood collection vessel such that the blood metering device is in fluid communication with the blood collection vessel;
inputting a predetermined fill volume to the blood metering device;
determining a gas pressure in the collection vessel with the valve closed;
determining, based on the inputted predetermined fill volume and determined gas pressure, a target gas pressure in the collection vessel at the predetermined fill volume;
determining, based on the determined target gas pressure, an expected amount of time to reach the predetermined fill volume;
closing the valve when the expected amount of time to reach the predetermined fill volume is reached;
continuously determining a gas pressure in the collection vessel;
continuously comparing the determined gas pressure in the collection vessel with the determined target gas pressure; and
closing the valve when the determined gas pressure is equal to the determined target gas pressure, wherein the determined target gas pressure in the collection vessel indicates that a target volume of blood has entered the collection vessel.

9. The method of claim 8, wherein a channel is defined in the housing of the control unit and connects the blood flow conduit and a pressure sensor disposed within the electronic compartment of the housing.

10. The method of claim 9, wherein the channel allows the pressure sensor to measure the gas pressure in the collection vessel when the valve is closed, which stops the blood from flowing through the blood metering device and to the collection vessel.

11. A blood collection system comprising:
a flow component comprising a flow channel extending between a first end and a second end, the flow channel having a T-junction;
a butterfly needle connected to the first end of the flow component;
a valve disposed between the butterfly needle and the T-junction of the flow channel;
a pressure sensor disposed in a channel adjacent to and in fluid communication with the T-junction; and
a blood metering device connected to the second end of the flow component, the blood metering device comprising;
a control unit, the control unit including a housing defining a blood flow conduit for blood from a patient to flow therethrough and an electronic module disposed within an electronic compartment of the housing;
a barrel configured to align with a neck of a collection vessel for receiving the neck therewithin; and
an adapter including a luer connector at a first end portion thereof for coupling with the control unit and a second end portion thereof for coupling with the barrel.

12. The blood collection system of claim 11, wherein a connection cavity is defined within the housing of the control unit for engaging and coupling with the adapter.

13. The blood collection system of claim 11, wherein the blood flow conduit defines a passageway therewithin for the blood from the patient to travel therethrough and to the collection vessel.

14. The blood collection system of claim 11, wherein the electronic module includes a printed circuit board for controlling components in the blood metering device, the pressure sensor connected to the printed circuit board, and a user input control.

15. The blood collection system of claim 11, wherein the flow channel is defined in the housing of the control unit and connects the blood flow conduit and the pressure sensor.

16. The blood collection system of claim 11, wherein the control unit, barrel, and adapter are all connected and in fluid communication with each other such that blood from the patient flows to the collection vessel.

17. The blood collection system of claim 15, wherein the flow channel allows the pressure sensor to measure a gas pressure at the T-junction.

18. A method for determining an accurate blood fill volume in a collection vessel, the method comprising:
providing a blood collection system comprising:
a flow component comprising a flow channel extending between a first end and a second end, the flow component comprising a T-junction;
a butterfly needle connected to the first end of the flow component;
a valve disposed between the butterfly needle and the T-junction of the flow component;
a pressure sensor connected to the flow component at the T-junction; and
a blood metering device connected to the second end of the flow component, the blood metering device comprising;
a control unit, the control unit including a housing defining a blood flow conduit for blood from a patient to flow therethrough and an electronic module disposed within an electronic compartment of the housing;
a barrel configured to align with a neck of a blood collection vessel to receive the neck therewithin; and
an adapter including a luer connector at a first end portion thereof for coupling with the control unit and a second end portion thereof for coupling with the barrel;
connecting the blood metering device to the blood collection vessel such that the blood metering device is in fluid communication with the blood collection vessel;
inputting a predetermined fill volume to the blood metering device;
determining a target gas pressure in the collection vessel at the predetermined fill volume;
opening the valve to allow blood drawn from a patient to flow;
measuring pressure at the T-junction of the flow component as the blood flows therethrough;
determining gas pressure in the collection vessel based on the measured pressure at the T-junction of the flow component;
comparing the determined gas pressure in the collection vessel with the determined target gas pressure; and
closing the valve when the determined gas pressure is equal to the determined target gas pressure, wherein the determined target gas pressure in the collection vessel indicates that a target volume of blood has entered the collection vessel.

19. The method of claim 18, wherein a channel is defined in the housing of the control unit and connects the blood flow conduit and a pressure sensor disposed within the electronic compartment of the housing.

20. The method of claim 19, wherein the channel allows the pressure sensor to measure the gas pressure at the T-junction.
